(19) United States
(12) Patent Application Publication
(10) Pub. No.: US 2004/0193132 A1
(43) Pub. Date: Sep. 30, 2004

(54) Disposable pamper with a detachable plastic feces container

(76) Inventor: Athenia Yvonne King, Philadelphia, PA (US)

Correspondence Address: Athenia Yvonne Wright'Campbell, 2022 S. Larry St., Philadelphia, PA, 19142, US

(21) Appl. No.: 10/319,350

(22) Filed: Mar. 28, 2003

(51) Int. Cl. A61F 13/15; A61F 13/20

(52) U.S. Cl. 604/385.19

(57) ABSTRACT

A pamper used in the normal fashion: with the exception of:

(4) Is an inside oval gasket covered by a stay dry lining. This horizontal gasket helps to hold the flat shape.

(5) Is a soft flexible O-ring, that is place in the center of the pamper, about 3″ to 4″ in width and vertically, that it

(6) A light soft flexible rubber ring, connected to a plastic detachable feces container. (About 8″ in length, and 3″ to 4″ in width.

(7) An outer oval gasket, (horizontal) with two small outer tabs, to pull detachable container out.

(8) Detachable plastic feces container.

### CROSS-REFERENCE TO RELATED APPLICATIONS

Not applicable.

### BACKGROUND-FIELD OF INVENTION

This invention relates to a plastic detachable container, which is inserted in the middle Of a disposable pamper, for disposal of fecal waste matter.

The pamper is used in the normal matter, except for an insertion of a 360 degree Circle put into the middle of the pamper, about 3″ horizontally and 4″ vertical made with A very light flexible rubber ring, which is molded around the circumference of the circle, Held in place by two large oval Gaskets. The circle is inserted about 10-14 inches from Flexible Round rubber ring, the outer gasket sides are on the inside to hold the shape. So It's Easier for the feces to be expelled while the patient is lying down.

There are two tabs on the outside, which are bend down to aid in the removal of the feces Container.

The container can be pulled out with a gentle pull, the feces is Contained in the plastic container. This makes it easier to clean, and dispose of, in the Proper manner.

### SUMMARY

This invention will work out really nice for people working in a nursing home, or in a Hospital setting. Families with elderly parents at home will find this invention most Helpful, the clean up part of the job is the hardest. With this invention the feces Is contained in the container, which will cut down allot of stress to you, as well as the Patient. The patient's skin will not break down with bedsores. Just detach the Container, and wash the patient with soap and water.

### DRAWING

FIG. 1. Is a perspective inside view?

FIG. 2. Is a perspective back view of the pamper

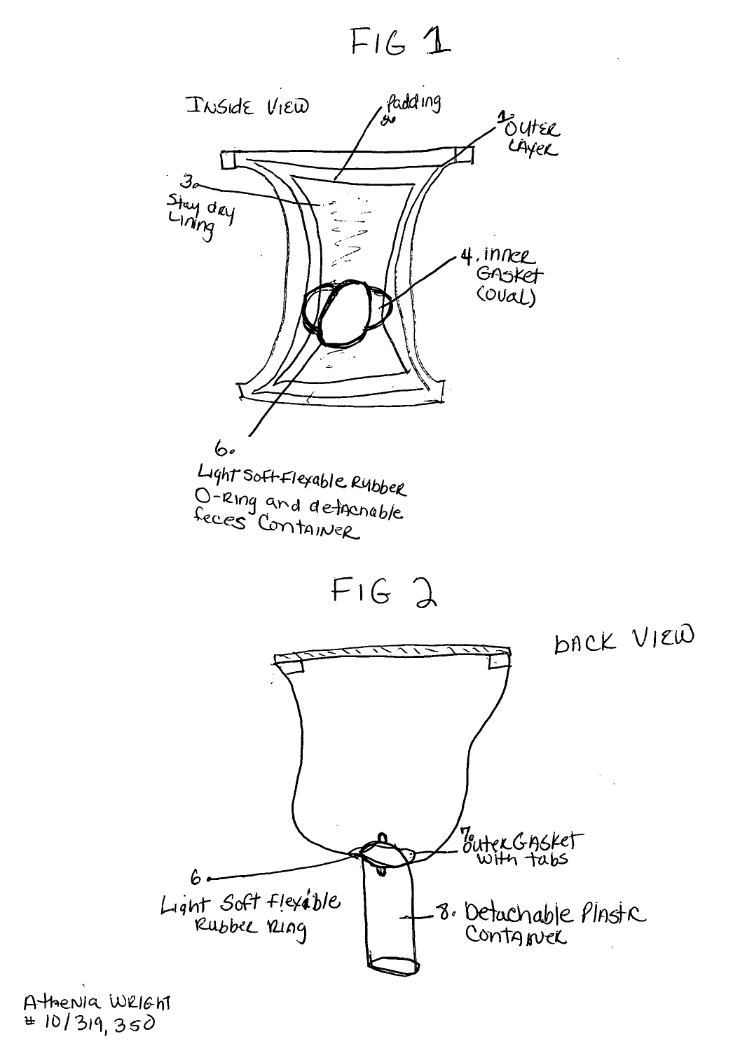

### REFERENCE NUMERALS

1) Outer layer

2) Padding

3) Stay dry lining

4) Inside gasket

5) O-ring

6) Soft flexible rubber ring

7) Outer gasket

8) Detachable plastic feces container

1. I claim that portions 4-8, what is claimed; is the disposable pamper, comprising of:
A. Detachable plastic feces container, the hold fecal waste
B. One inner gasket (oval) to hold the detachable container
C. One flexible O-ring
D. One outer gasket
